(19) United States
(12) Patent Application Publication
(10) Pub. No.: US 2022/0282293 A1
(43) Pub. Date: Sep. 8, 2022

(54) ENTEROBACTER CHENGDUENSIS FOR PRODUCING NICOTINAMIDE MONONUCLEOTIDE AND APPLICATION THEREOF

(71) Applicant: HOBOOMLIFE BIO-TECHNOLOGY (SHENZHEN) CO., LTD., Shenzhen (CN)

(72) Inventors: Liqing Zhao, Shenzhen (CN); Jiansheng Chen, Shenzhen (CN); Zhigang Duan, Shenzhen (CN); Haichao Zhang, Shenzhen (CN)

(21) Appl. No.: 17/520,714

(22) Filed: Nov. 7, 2021

(30) Foreign Application Priority Data
Mar. 8, 2021 (CN) 202110250748.7

(51) Int. Cl. C12P 19/30; C12M 1/26

(52) U.S. Cl. CPC C12P 19/30; C12M 33/10; C12N 1/205

(57) ABSTRACT

The present disclosure provides an Enterobacter chengduensis for producing NMN and application thereof, and relates to the technical field of screening and application of strains. The Enterobacter chengduensis of the present disclosure is determined as a Gram-negative bacterium and belongs to the genus Enterobacter chengduensis. When the Enterobacter chengduensis is used for producing the NMN by fermentation with the nicotinamide as a substrate, the yield of the NMN can reach 67.66 μM, namely 22.6 mg/L, at 15 minutes, indicating that the wild strain has a strong activity to synthesize the NMN, the dependence on Nampt during synthesis of the NMN is reduced, and a high large-scale application prospect is achieved.

### CROSS-REFERENCE TO RELATED APPLICATION

The present application claims the benefit of Chinese Patent Application No. 202110250748.7 filed on Mar. 8, 2021, the disclosure of which is hereby incorporated by reference in their entirety.

### REFERENCE TO SEQUENCE LISTING

The Sequence Listing is submitted as an ASCII formatted text file via EFS-Web, with a file name of “Sequence_listing.TXT”, a creation date of Nov. 4, 2021, and a size of 2,025 bytes. The Sequence Listing filed via EFS-Web is part of the specification and is incorporated in its entirety by reference herein.

### TECHNICAL FIELD

The present disclosure belongs to the technical field of screening and application of strains, and specifically relates to an Enterobacter chengduensis for producing nicotinamide mononucleotide and application thereof.

### BACKGROUND

Nicotinamide nononucleotide (NMN for short) is an organic molecule and also a nucleotide and has the effects of reversing aging and prolonging life.

At present, enzymatic reactions are mostly used to achieve large-scale synthesis of the NMN, but the enzymatic reactions are high in costs, harsh in reaction conditions and unstable in production processes; products of different batches have different indexes, and the reaction capacity is low. In recent years, there are also methods for producing the NMN by biological fermentation means, but natural niacinamide phosphoribosyltransferase (Nampt) has low enzymatic activity, the catalytic time is long, the cost is high, the yield is low, and large-scale industrialized production is difficult to achieve, so that large-scale application of the NMN is limited. For this reason, the Chinese patent CN201811606780.9 discloses a method for producing an NMN by fermentation of a recombinant Escherichia coli. However, the cost of a genetically modified recombinant strain is relatively high, and the biological safety is questionable, especially when the strain is applied to cosmetics, food or medicine.

### SUMMARY

In view of this situation, an objective of the present disclosure is to provide an Enterobacter chengduensis for producing NMN and having high production activity and application thereof. The Enterobacter chengduensis can catalyze the production of the NMN from nicotinamide (Nam), and a foundation is laid for subsequent industrial production.

To achieve the foregoing objectives, the present disclosure provides the following technical solutions:

The present disclosure provides an Enterobacter chengduensis 2021T4.7 for producing NMN, and the Enterobacter chengduensis 2021T4.7 has a biological deposit number of CGMCC No. 21695.

Preferably, a 16S rDNA sequence of the Enterobacter chengduensis 2021T4.7 is shown in SEQ ID NO: 1.

The present disclosure also provides application of the Enterobacter chengduensis 2021T4.7 in production of NMN.

The present disclosure also provides a method for producing NMN by using the Enterobacter chengduensis 2021T4.7, and the method includes the following steps:

(1) performing fermentation enrichment on the Enterobacter chengduensis 2021T4.7 in a fermentation culture medium with the Nam as an inducer, and after fermentation, performing centrifugation and resuspension in a PBS buffer to obtain a bacterial liquid, where the fermentation culture medium includes the following components by mass percent: 0.25% to 1.5% glucose, 0.25% to 1.5% tryptone, 0 to 1.5% KH₂PO₄ and 0 to 0.15% MgSO₄·7H₂O, and a pH value is 5 to 10; and

(2) performing a fermentation reaction by using the bacterial liquid obtained in step (1) with the Nam as a substrate to obtain the NMN.

Preferably, in step (1), the mass percentage of the inducer Nam is 0 to 1.25%.

Preferably, in step (1), the pH value of the fermentation culture medium is 7.0.

Preferably, in step (1), a fermentation enrichment temperature is 25° C. to 40° C., and time is 12-24 hours.

Preferably, a mother liquid is further involved in the fermentation reaction in step (2), a volume ratio of the mother liquid to the bacterial liquid is 1:1, and the mother liquid includes the following components: a 50 mM Tris-Hcl buffer, BSA with a mass concentration of 0.02%, 12 mM MgCl₂, 2 mM ATP, 2 mM DTT and 40 μM PRPP.

Preferably, in step (2), a concentration of the substrate Nam is 100-1,000 μM.

Preferably, in step (2), a fermentation reaction temperature is 37° C., and time is 15 minutes.

The present disclosure provides an Enterobacter chengduensis 2021T4.7 for producing NMN, the Enterobacter chengduensis is obtained by collecting soil near a sewer of a factory for producing NMN-related products, performing preliminary screening and secondary screening and selecting a strain with a strong ability to convert the Nam into the NMN, the strain is identified and deposited, and it is determined that the Enterobacter chengduensis 2021T4.7 is a Gram-negative bacterium and belongs to the genus Enterobacter chengduensis.

When the Enterobacter chengduensis 2021T4.7 is used for producing the NMN by fermentation with the Nam as a substrate, the yield of the NMN can reach 67.66 μM, namely 22.6 mg/L, at 15 minutes, indicating that the wild strain has a strong activity to synthesize the NMN, a Nampt isoenzyme can be provided, and the dependence on Nampt during synthesis of the NMN is reduced. The Enterobacter chengduensis of the present disclosure is a wild bacterium, and compared with a recombinant bacterium for producing the NMN by fermentation, the Enterobacter chengduensis is lower in cost and higher in biological safety.

### Biological Deposit Information

The Enterobacter chengduensis 2021T4.7 was deposited at the China General Microbiological Culture Collection Center (CGMCC) on Jan. 21, 2021, the specific address is Institute of Microbiology, CAS, Building 3, Lane 1, West Beichen Road, Chaoyang District, Beijing 100101, China, and the deposit number is CGMCC No. 21695.

### BRIEF DESCRIPTION OF THE DRAWINGS

FIG. 1 shows a standard curve of an NMN concentration and a fluorescence intensity;

FIG. 2 is a morphological diagram of an Enterobacter chengduensis;

FIG. 3 shows staining results of the Enterobacter chengduensis after Gram staining;

FIG. 4 is a diagram showing the influence of optimization of the content of glucose in a fermentation culture medium in step (1) on the yield of an NMN;

FIG. 5 is a diagram showing the influence of optimization of the content of tryptone in the fermentation culture medium in step (1) on the yield of the NMN;

FIG. 6 is a diagram showing the influence of optimization of the content of KH₂PO₄ in the fermentation culture medium in step (1) on the yield of the NMN;

FIG. 7 is a diagram showing the influence of optimization of the content of MgSO₄·7H₂O in the fermentation culture medium in step (1) on the yield of the NMN;

FIG. 8 is a diagram showing the influence of optimization of the content of an inducer Nam in step (1) on the yield of the NMN;

FIG. 9 is a diagram showing the influence of optimization of an initial pH value of the fermentation culture medium in step (1) on the yield of the NMN;

FIG. 10 is a diagram showing the influence of optimization of the fermentation enrichment temperature in step (1) on the yield of the NMN;

FIG. 11 is a diagram showing the influence of optimization of the fermentation enrichment time in step (1) on the yield of the NMN; and

FIG. 12 is a diagram showing the influence of optimization of the content of a substrate Nam in step (2) on the yield of the NMN under optimal fermentation enrichment conditions in step (1).

### DETAILED DESCRIPTION

The present disclosure provides an Enterobacter chengduensis 2021T4.7 for producing NMN, and the Enterobacter chengduensis 2021T4.7 has a biological deposit number of CGMCC No. 21695.

The Enterobacter chengduensis 2021T4.7 of the present disclosure is a Gram-negative bacterium, a 16S rDNA sequence is preferably shown in SEQ ID NO: 1: TTACTGGGCGTAAGCGCACGCAGGCGGTCTGTCAAGTCGGATGTGAAATCCCCG GGCTCAACCTGGGAACTGCATTCGAAACTGGCAGGCTAGAGTCTTGTAGAGGGG GGTAGAATTCCAGGTGTAGCGGTGAAATGCGTAGAGATCTGGAGGAATACCGGT GGCGAAGGCGGCCCCCTGGACAAAGACTGACGCTCAGGTGCGAAAGCGTGGGG AGCAAACAGGATTAGATACCCTGGTAGTCCACGCCGTAAACGATGTCGACTTGG AGGTTGTGCCCTTGAGGCGTGGCTTCCGGAGCTAACGCGTTAAGTCGACCGCCTG GGGAGTACGGCCGCAAGGTTAAAACTCAAATGAATTGACGGGGGCCCGCACAAG CGGTGGAGCATGTGGTTTAATTCGATGCAACGCGAAGAACCTTACCTACTCTTGA CATCCAGAGAACTTTCCAGAGATGGATTGGTGCCTTCGGGAACTCTGAGACAGGT GCTGCATGGCTGTCGTCAGCTCGTGTTGTGAAATGTTGGGTTAAGTCCCGCAACG AGCGCAACCCTTATCCTTTGTTGCCAGCGGTTAGGCCGGGAACTCAAAGGAGACT GCCAGTGATAAACTGGAGGAAGGTGGGGATGACGTCAAGTCATCATGGCCCTTA CGAGTAGGGCTACACACGTGCTACAATGGCGCATACAAAGAGAAGCGACCTCGC GAGAGCAAGCGGACCTCATAAAGTGCGTCGTAGTCCGGATTGGAGTCTGCAACT CGACTCCATGAAGTCGGAATCGCTAGTAATCGTAGATCAGAATGCTACGGTGAA TACGTTCCCGGGCCTTGTACACACCGCCCGTCACACCATGGGAGTGGGTTGCAAA AGAAGTAGGTAGCTTAACCTTCGGGAGGGCGCTTACCACTTTGTGATTCATGACT GGGGTGAA, the similarity between the sequence and available sequences of known species in an NCBI database is 99%, and it is determined that the strain belongs to the genus Enterobacter.

The Enterobacter chengduensis 2021T4.7 of the present disclosure is screened and separated from soil near a sewer of a factory of Bontac Bioengineering (Shenzhen) Co., Ltd., and after preliminary screening and secondary screening, a strain with a strong ability to convert Nam into the NMN is selected.

The present disclosure also provides application of the Enterobacter chengduensis 2021T4.7 in production of NMN. The Enterobacter chengduensis 2021T4.7 of the present disclosure has the ability to decompose the Nam and produce the NMN and can be used for industrial production of the NMN.

The present disclosure also provides a method for producing NMN by using the Enterobacter chengduensis 2021T4.7, and the method includes the following steps:

(1) performing fermentation enrichment on the Enterobacter chengduensis 2021T4.7 in a fermentation culture medium with the Nam as an inducer, and after fermentation, performing centrifugation and resuspension in a PBS buffer to obtain a bacterial liquid, where the fermentation culture medium includes the following components by mass percent: 0.25% to 1.5% glucose, 0.25% to 1.5% tryptone, 0 to 1.5% KH₂PO₄ and 0 to 0.15% MgSO₄·7H₂O, and a pH value is 5 to 10; and

(2) performing a fermentation reaction by using the bacterial liquid obtained in step (1) with the Nam as a substrate to obtain the NMN.

In the present disclosure, the fermentation enrichment is performed on the Enterobacter chengduensis 2021T4.7 in a fermentation culture medium with the Nam as an inducer, and after fermentation, the centrifugation and the resuspension in a PBS buffer are performed. The fermentation culture medium includes the following components by mass percent: 0.25% to 1.5% glucose, 0.25% to 1.5% tryptone, 0 to 1.5% KH₂PO₄ and 0 to 0.15% MgSO₄·7H₂O. A pH value is 5 to 10. The fermentation culture medium in the present disclosure is obtained by using the glucose as a carbon source, the tryptone as a nitrogen source and the Nam as an inducer. The mass percentage of the glucose in the fermentation culture medium is preferably 1%, the mass percentage of the tryptone is preferably 1.25%, the mass percentage of the KH₂PO₄ is preferably 0.75%, the mass percentage of the MgSO_{4·7}H₂O is preferably 0.025%, and the mass of the inducer Nam is preferably 0 to 1.25% of the mass of the fermentation culture medium, more preferably 1%. The pH value of the fermentation culture medium in the present disclosure is preferably 7. A preparation method of the fermentation culture medium in the present disclosure is not particularly limited and preferably includes uniformly mixing the components above. Sources of the components of the fermentation culture medium in the present disclosure are not particularly limited. The Nam included in the fermentation culture medium in the present disclosure achieves an inducing effect and can be used for amplifying the Enterobacter.

In the present disclosure, when the fermentation enrichment is performed, the inoculation volume of the Enterobacter is preferably 0.1% to 2% of the volume of the fermentation culture medium, more preferably 1.5%. In the present disclosure, the fermentation enrichment temperature is preferably 25° C. to 40° C., more preferably 37° C. The fermentation enrichment time is preferably 12-24 hours, more preferably 20 hours. In the present disclosure, shaking is preferably performed in the fermentation enrichment process, and the shaking frequency is preferably 200 rpm.

In the present disclosure, after the fermentation, the centrifugation and the resuspension in a PBS buffer are performed. The centrifugation is preferably performed at a rotation speed of 4,000 rpm at 4° C. for 20 minutes. In the present disclosure, a method for performing resuspension in a PBS buffer is not particularly limited. The resuspension can be performed by using a conventional method in the art. The OD₆₀₀ of the bacterial liquid after the resuspension is preferably adjusted to 1.5.

After the bacterial liquid is obtained, the Nam is used as a substrate in the present disclosure, and the bacterial liquid is used to perform a fermentation reaction to obtain the NMN. The fermentation reaction in the present disclosure is preferably performed in a system obtained by mixing the bacterial liquid and a mother liquid at a volume ratio of 1:1. The mother liquid preferably includes the following components: a 50 mM Tris-Hcl buffer, BSA with a mass concentration of 0.02%, 12 mM MgCl₂, 2 mM ATP, 2 mM DTT and 40 μM PRPP. When the fermentation reaction in the present disclosure is performed, the concentration of the substrate Nam is preferably 100-1,000 μM, more preferably 400 μM. The reaction is preferably performed at 37° C. for 15 minutes.

The present disclosure also provides a primer pair for identifying the Enterobacter chengduensis 2021T4.7. The primer pair includes a forward primer 516f and a reverse primer 1540r. A nucleotide sequence of the forward primer 516f is shown in SEQ ID NO: 2: TGCCAGCAGCCGCGGTA, and a nucleotide sequence of the reverse primer 1540r is shown in SEQ ID NO: 3: AGGAGGTGATCCAGCCGCA.

The Enterobacter chengduensis for producing NMN and application thereof provided in the present disclosure are described in detail below with reference to examples, but it should not be understood that the protection scope of the present disclosure is limited thereto.

Unless otherwise specified, components and sources of culture media in the examples of the present disclosure are all common commercial products.

### EXAMPLE 1 SCREENING AND IDENTIFICATION OF STRAINS

### 1. Soil Collection and Pretreatment

Soil near a sewer of a factory of Bontac Bioengineering (Shenzhen) Co., Ltd. was collected with a sampling depth of 5-10 cm. 1 g of a soil sample was weighed and added into a 50 ml conical flask, 20 ml of physiological saline was added, the conical flask was placed in a shaker and thoroughly shaken uniformly, and then the conical flask was taken out for standing and later use.

### 2. Enrichment Culture

1 ml of a supernatant of a soil suspension obtained after pretreatment was sucked, inoculated into an enrichment culture medium (10 ml/50 ml conical flask) and then cultured in a constant-temperature incubator at 37° C. and 150 r/min for 12 hours. The enrichment culture medium included 2 g/L Nam, 5 g/L glucose, a 5 g/L yeast extract, 5 g/L peptone, 14 g/L K₂HPO₄·3H₂O, 5.2 g/L KH₂PO₄ and 2 g/L MgSO₄·7H₂O, and the pH of the enrichment culture medium was 7.0.

### 3. Screening

Preliminary screening: An enrichment liquid was subjected to gradient dilution with the physiological saline at different levels, and bacterial liquids diluted to gradients of 10⁻⁴, 10⁻⁵ and 10⁻⁶ are used for preliminary screening of strains. 100 μl of each diluted liquid was taken, spread on a preliminary screening plate separation culture medium and then cultured overnight, and single colonies on the plate were selected on the next day (bacteria with different shapes, colors and sizes were selected) and transferred into a 96 deep-well cell plate (same as the preliminary screening plate separation culture medium) for shaking culture at a rotation speed of 600 r/min for 12 hours. The content of NMN in each sample was detected by using a microplate reader detection method. Strains with a high NMN content were selected based on fluorescence intensity and then preserved in a glycerol tube. The preliminary screening plate separation culture medium included 2 g/L Nam, a 5 g/L yeast extract, 5 g/L peptone, 14 g/L K₂HPO₄.3H₂O, 5.2 g/L KH₂PO₄ and 14 g/L MgSO₄·7H₂O, and the pH of the preliminary screening plate separation culture medium was 7.0.

Secondary screening: The strains obtained after preliminary screening were inoculated into a nutrient agar culture medium (10 ml/50 ml conical flask) at 1% for fermentation subculture, the OD₆₀₀ value of a fermentation liquid was determined, an enzymatic conversion reaction experiment was performed, the fluorescence intensity of a system after NMN derivatization was determined, and strains with a high NMN yield were obtained after secondary screening based on indexes of a product concentration and a conversion rate. The nutrient agar culture medium included 12.5 g/L agar powder, 10 g/L tryptone, a 5 g/L yeast extract and 5 g/L NaCl, and the pH of the nutrient agar culture medium was 7.0.

### 4. Enzymatic Conversion Reaction Experiment

1 ml of the bacterial liquids obtained after the enrichment culture were taken, first centrifuged to remove culture solutions in supernatants and then resuspended in a PBS buffer (the Nam in a fermentation culture medium was removed), the OD₆₀₀ value was determined, and the concentration of the bacterial liquids was appropriate when the OD₆₀₀ was adjusted to 1.5. The Nam was taken as a substrate, a 50 mM Tris-Hcl buffer, BSA with a mass concentration of 0.02%, 12 mM MgCl₂, 2 mM ATP, 2 mM DTT, 40 μM PRPP and 80 μM Nam were taken in a total of 12.5 μl, and 12.5 μl of the bacterial liquids were taken, sequentially added into a black 96-well Elisa plate (special for fluorescence measurement) and shaken and mixed uniformly for a reaction at 37° C. for 15 minutes.

### 5. Determination of the Yield of the NMN

Since the NMN has fluorescence signals at an excitation wavelength of 382 nm and an emission wavelength of 445 nm, the ability of different strains to produce the NMN can be compared.

After an enzymatic conversion reaction was completed, 10 μl of 20% acetophenone and 10 μl of 2 M KOH were added into a reaction well of the 96-well Elisa plate and shaken and mixed uniformly to produce a white precipitate, and the white precipitate was placed into a metal bath at 0° C. for a reaction for 10 minutes. The Elisa plate was taken out from the metal bath, and 45 μl of 88% formic acid was added into the metal bath for a reaction at 70° C. for 5 minutes. After cooling, the fluorescence intensity was measured at the excitation wavelength of 382 nm and the emission wavelength of 445 nm by using a microplate reader, and the gain value was set to 70.

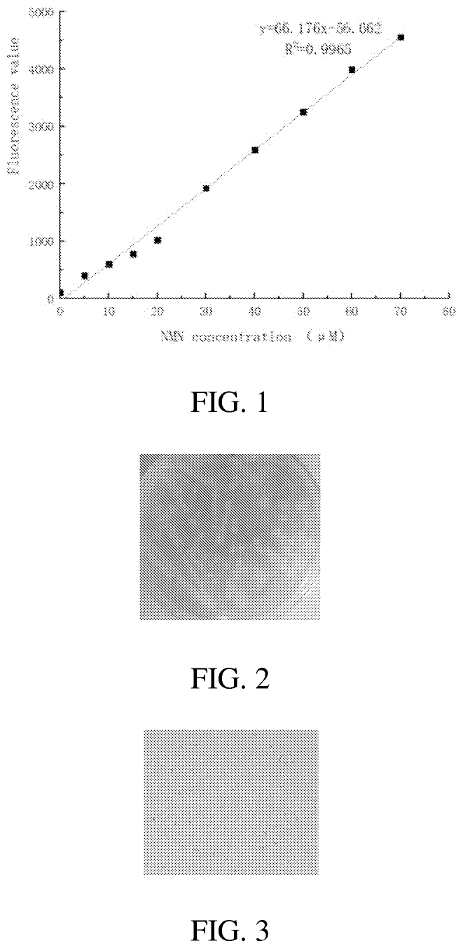

In this example, under the same reaction system of the “enzymatic conversion reaction experiment”, the NMN was added to achieve different concentrations (0, 5 μM, 10 μM, 15 μM, 20 μM, 25 μM, 30 μM, 35 μM, 40 μM, 45 μM, 50 μM, 55 μM, 60 μM, 70 μM, and 80 μM), the fluorescence intensity was measured at the excitation wavelength of 382 nm and the emission wavelength of 445 nm by using the microplate reader, the gain value was set to 70, and a standard curve of the NMN concentration and the fluorescence intensity was obtained and shown in FIG. 1. The x-axis represents the NMN concentration, the y-axis represents the fluorescence intensity, y=66.176x-56.662, and R²=0.9965. According to this standard curve, corresponding concentrations of the NMN under different fluorescence intensities could be obtained.

### 6. Strain Identification

Morphological observation: The strains preserved in the glycerol tube were taken for activation culture in an LB liquid culture medium (37° C., 180 rpm, shaking culture for 12 hours), a bacterial liquid was taken and spread on an LB plate separation culture medium to culture and isolate a single colony, the state of the single colony on a solid plate was observed, and results were shown in FIG. 2. The isolated and purified single colony was taken, cultured overnight and then stained by using a Gram staining method, Gram staining results were observed by using a microscope, and results were shown in FIG. 3. This strain was a Gram-negative bacterium and belongs to the genus Enterobacter chengduensis.

A bacterial genomic DNA rapid extraction kit purchased from Sangon Biotech (Shanghai) Co., Ltd. was used to extract DNA from a chromosome, and PCR was used for amplification, followed by agarose gel electrophoresis analysis. An amplified DNA sequence was about 1,000 bp in length, a PCR amplified product was sent to Guangzhou Ige Biotechnology Co., Ltd. for 16S rDNA sequencing, sequencing results were shown in SEQ ID NO: 1, and it was identified that the strain belonged to the genus Enterobacter chengduensis and was named Enterobacter chengduensis 2021T4.7.

Forward and reverse primers of PCR were:
516f: TGCCAGCAGCCGCGGTA,
and
1540r: AGGAGGTGATCCAGCCGCA.

### EXAMPLE 2 INFLUENCE OF OPTIMIZATION OF FERMENTATION ENRICHMENT CONDITIONS OF ENTEROBACTER CHENGDUENSIS 2021T4.7 ON THE YIELD OF NMN

When the Enterobacter chengduensis 2021T4.7 was subjected to fermentation enrichment in a fermentation culture medium, concentrations of various components of the fermentation culture medium (including the concentration of glucose, the concentration of tryptone, the concentration of KH₂PO₄ and the concentration of MgSO₄·7H₂O), the initial pH value of the fermentation culture medium, the concentration of an inducer Nam and the fermentation enrichment temperature and time were sequentially changed, other conditions were not changed, and shaking culture was performed in a constant-temperature shaker at 200 rpm. Then fermentation was performed under the conditions of the “enzymatic conversion reaction experiment” in Example 1, and after the fermentation was completed, the OD₆₀₀ and the enzymatic activity were determined.

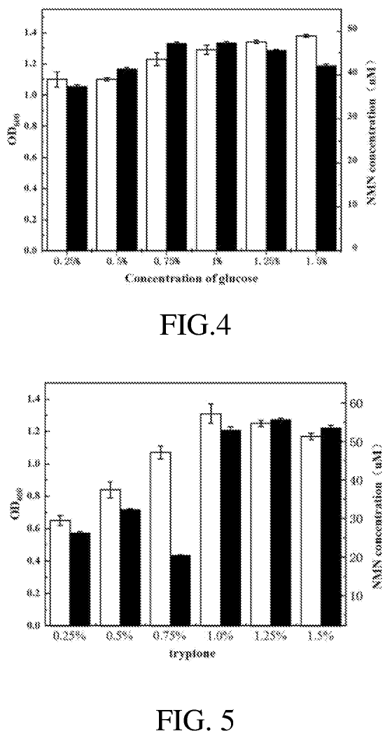

1. When different concentrations of the glucose (0.25%, 0.5%, 0.75%, 1%, 1.25% and 1.5% by mass fraction) were added into the fermentation culture medium and other components were not changed, results were shown in FIG. 4. When the concentration of the glucose was 1%, the yield of the NMN was the highest and was 47.36 μM.

2. When different concentrations of the tryptone (0.25%, 0.5%, 0.75%, 1%, 1.25% and 1.5% by mass fraction) and 1% glucose were added into the fermentation culture medium and other components were not changed, results were shown in FIG. 5. When the concentration of the tryptone was 1.25%, the yield of the NMN was the highest and was 55.67 μM.

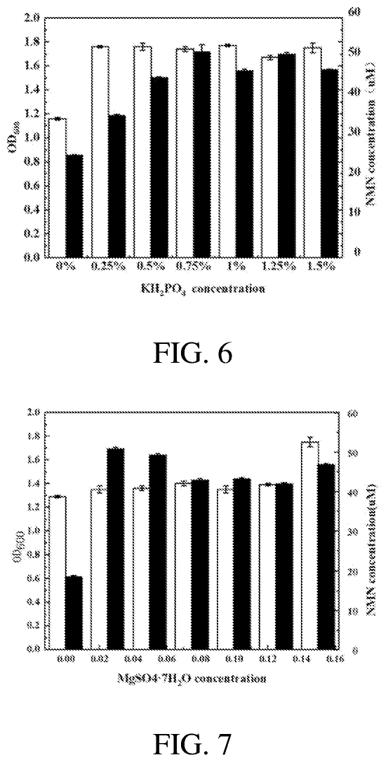

3. When different concentrations of the KH₂PO₄ (0, 0.25%, 0.5%, 0.75%, 1%, 1.25% and 1.5% by mass fraction), 1% glucose and 1.25% tryptone were added into the fermentation culture medium and other components were not changed, results were shown in FIG. 6. When the content of the KH₂PO₄ was 0.75%, the yield of the NMN was the highest and was 51.43 μm.

4. When different concentrations of the MgSO₄·7H₂O (0, 0.025%, 0.05%, 0.075%, 0.1%, 0.125% and 0.15% by mass fraction), 1% glucose, 1.25% tryptone and 0.75% KH₂PO₄ were added into the fermentation culture medium and other conditions were not changed, results were shown in FIG. 7. When the content of the MgSO₄·7H₂O was 0.025%, the yield of the NMN was the highest and was 50.93 μM.

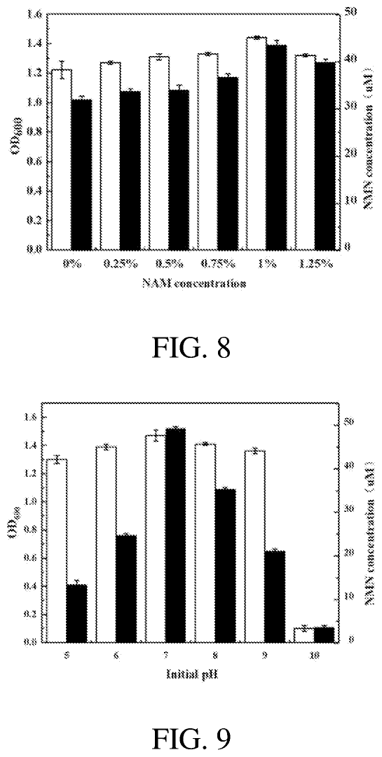

5. When different concentrations of the inducer Nam (0%, 0.25%, 0.5%, 0.75%, 1.0% and 1.25% by mass fraction), 1% glucose, 1.25% tryptone, 0 .75% KH₂PO₄ and 0.025% MgSO₄·7H₂O were added into the fermentation culture medium and other conditions were not changed, results were shown in FIG. 8. When the added amount of the inducer Nam was 1%, the yield of the NMN was the highest and was 43.57 μM.

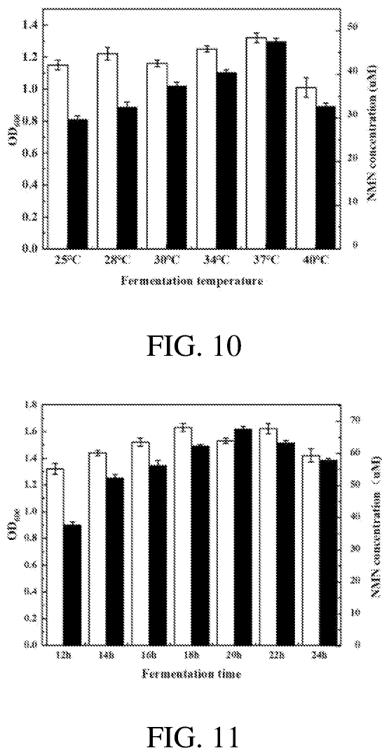

6. When a fermentation culture medium containing 1% of the glucose, 1.25% of the tryptone, 0.75% of the KH₂PO₄, 0.025% of the MgSO₄·7H₂O and 1% of the inducer Nam was used, the initial pH value (5.0, 6.0, 7.0, 8.0, 9.0 and 10.0) of the fermentation culture medium and the fermentation enrichment temperature (25° C., 28° C., 30° C., 34° C., 37° C. and 40° C.) and time (12 hours, 14 hours, 16 hours, 18 hours, 20 hours, 22 hours and 24 hours) were sequentially changed and other conditions were not changed, results were shown in FIG. 9 to FIG. 11. When the initial pH value of the fermentation culture medium was 7.0, the yield of the NMN was the highest and was 49.31 μM. When the fermentation enrichment temperature was 37° C., the yield of the NMN was the highest and was 47.52 μM. When the fermentation enrichment time was 20 hours, the yield of the NMN was the highest and was 67.66 μM.

### EXAMPLE 3 INFLUENCE OF THE CONCENTRATION OF A SUBSTRATE NAM ON THE YIELD OF NMN

Under optimal fermentation enrichment conditions of the Enterobacter chengduensis 2021T4.7 in Example 2 (that is to say, the fermentation culture medium included 1% glucose, 1.25% tryptone, 0.75% KH₂PO₄, 0.025% MgSO₄·7H₂O and 1% inducer Nam, the pH value was 7.0, the fermentation temperature was 37° C., and the fermentation time was 20 hours), fermentation was performed by only changing the concentration of the substrate Nam in the enzymatic conversion reaction experiment in Example 1 (100 μM, 200 μM, 300 μM, 400 μM, 500 μM, 600 μM, 700 μM, 800 μM, 900 μM and 1,000 μM), other conditions were not changed, and after the fermentation was completed, the OD₆₀₀ and the enzymatic activity were determined.

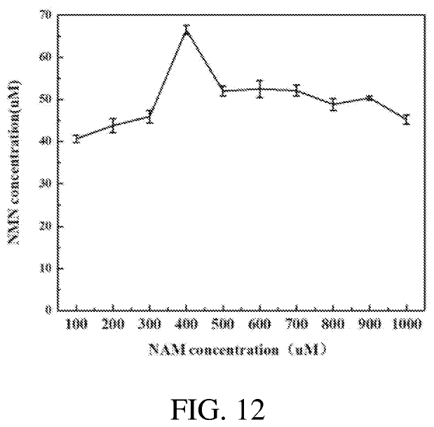

Results were shown in FIG. 12. When the concentration of the substrate Nam was 400 μM, the yield of the NMN was the highest and was 66.47 μM.

The foregoing descriptions are exemplary implementations of the present disclosure. It is to be noted that a person of ordinary skill in the art may make some improvements and modifications without departing from the principle of the present disclosure and such improvements and modifications shall fall within the protection scope of the present disclosure.

1. An Enterobacter chengduensis 2021T4.7 for producing a nicotinamide mononucleotide (NMN), wherein the Enterobacter chengduensis 2021T4.7 was deposited at China General Microbiological Culture Collection Center (CGMCC) with deposit number of CGMCC No. 21695.

2. The Enterobacter chengduensis 2021T4.7 according to claim 1, wherein a 16S rDNA sequence of the Enterobacter chengduensis 2021T4.7 is shown in SEQ ID NO: 1.

3. (canceled)

4. A method for producing NMN by using the Enterobacter chengduensis 2021T4.7 according to claim 1, comprising the following steps:
(1) performing fermentation enrichment on the Enterobacter chengduensis 2021T4.7 in a fermentation culture medium with nicotinamide as an inducer, and after fermentation, performing centrifugation and resuspension in a PBS buffer to obtain a bacterial liquid, wherein the fermentation culture medium comprises the following components by mass percent: 0.25% to 1.5% glucose, 0.25% to 1.5% tryptone, 0 to 1.5% KH₂PO₄ and 0 to 0.15% MgSO₄·7H₂O, and a pH value is 5 to 10; and
(2) performing a fermentation reaction by using the bacterial liquid obtained in step (1) with the nicotinamide as a substrate to obtain the NMN.

5. The method according to claim 4, wherein in step (1), the mass percentage of the inducer nicotinamide is 0 to 1.25%.

6. The method according to claim 5, wherein in step (1), the pH value of the fermentation culture medium is 7.0.

7. The method according to claim 4, wherein in step (1), a fermentation enrichment temperature is 25° C. to 40° C., and time is 12-24 hours.

8. The method according to claim 4, wherein a mother liquid is further involved in the fermentation reaction in step (2), a volume ratio of the mother liquid to the bacterial liquid is 1:1, and the mother liquid comprises the following components: a 50 mM Tris-Hcl buffer, BSA with a mass concentration of 0.02%, 12 mM MgCl₂, 2 mM ATP, 2 mM DTT and 40 μM PRPP.

9. The method according to claim 4, wherein in step (2), a concentration of the substrate nicotinamide is 100-1,000 μM.

10. The method according to claim 4, wherein in step (2), a fermentation reaction temperature is 37° C., and time is 15 minutes.


SEQUENCE LISTING (3 sequences)

SEQ ID NO: 1 - DNA, 932 bp, Enterobacter chengduensis
ttactgggcgtaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcggttaggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaa

SEQ ID NO: 2 - DNA, 17 bp, Artificial sequence
  synthetic
tgccagcagccgcggta

SEQ ID NO: 3 - DNA, 19 bp, Artificial sequence
  synthetic
aggaggtgatccagccgca